(19) United States
(12) Patent Application Publication
(10) Pub. No.: US 2009/0253599 A1
(43) Pub. Date: Oct. 8, 2009

(54) Crosslinking method and crosslinked polysaccharide made thereby

(75) Inventors: Kraig Luczak, Cranbury, NJ (US); Caroline Mabille, Paris (FR)

Correspondence Address: RHODIA, INC., 8 CEDAR BROOK DRIVE, CN 7500, CRANBURY, NJ, 08512, US

(73) Assignee: Rhodia Inc., Cranbury, NJ (US)

(21) Appl. No.: 12/384,641

(22) Filed: Apr. 7, 2009

Related U.S. Application Data
(60) Provisional application No. 61/123,364, filed on Apr. 7, 2008.
(60) Provisional application No. 61/135,560, filed on Jul. 22, 2008.

(51) Int. Cl. A61K 8/73; C08B 37/00; A61Q 5/02

(52) U.S. Cl. 510/121; 536/114

(57) ABSTRACT

A method for crosslinking a polysaccharide, includes the step of contacting particles of the polysaccharide with a titanium compound in an aqueous medium under conditions appropriate to intra-particulately crosslink the discrete particles.

### CROSS-REFERENCE TO RELATED APPLICATIONS

This application claims the benefit of U.S. Provisional Application Ser. No. 61/135,560, filed Jul. 22, 2008, and U.S. Provisional Application Ser. No. 61/123,364, filed Apr. 7, 2008, herein incorporated by reference.

### FIELD OF THE INVENTION

This invention relates to a crosslinking method and crosslinked polysaccharide made thereby.

### BACKGROUND OF THE INVENTION

Polysaccharides, including derivatized polysaccharides such as carboxylmethyl guar gum, hydroxypropyl guar gum, and hydroxypropyl trimethylammonium guar gum, are commercially available materials used in a variety of applications, including as ingredients in personal care compositions.

In the processing of such polysaccharides, it is sometimes desirable to form crosslinked particles of the polysaccharide that are relatively insoluble in water, in order to allow formation of an aqueous dispersion of polysaccharide particles that remains fluid and easily tractable. For example, guars are typically made by a “water-splits” process, wherein material, known as guar “splits”, derived from guar seeds undergoes reaction with a derivatizing agent in an aqueous medium. Borax (sodium tetra borate) is commonly used as a processing aid in the reaction step of the water-splits process to partially crosslink the surface of the guar splits and thereby reduces the amount of water absorbed by the guar splits during washing. The borate crosslinking takes place under alkaline conditions and is reversible allowing the product to hydrate under acidic conditions. Maintaining the moisture content of the derivatized splits at a relatively low level, typically a moisture content of less than or equal to about 90 percent by weight, simplifies handling and milling of the washed derivatized splits. In the absence of crosslinking, the moisture content of washed derivatized splits is relatively high and handling and further processing of the high moisture content splits is difficult. Prior to end-use application, for example, as a thickener in an aqueous personal care composition such as a shampoo, the crosslinked guar is typically dispersed in water and the boron crosslinking then reversed by adjusting the pH of the guar dispersion, to allow dissolution of the guar to form a viscous aqueous solution.

However, the use of borate crosslinking agents may be undesirable in some end-use applications due to evolving product regulatory requirements.

What is needed is an alternative to boron crosslinking as a process aid to simplify the manufacture and handling of polysaccharide thickeners, including derivatized polysaccharide thickeners, such as derivatized guars.

### SUMMARY OF THE INVENTION

In a first aspect, the present invention is directed to a method for crosslinking a polysaccharide, comprising contacting particles of the polysaccharide with a titanium compound in an aqueous medium under conditions appropriate to intra-particulately crosslink the discrete particles. The step of crosslinking the polysaccharide occurs before or after a wash step is performed on the polysaccharide particles, typically after a wash step is performed.

In a second aspect, the present invention is directed to a method for making crosslinked derivatized polysaccharides, comprising: (a) contacting particles of a polysaccharide with a titanium compound in an aqueous medium under conditions appropriate to intra-particulately crosslink the particles; (b) reacting, prior to or subsequent to the step of contacting the particles of polysaccharide with the titanium compound, the particles of polysaccharide with a derivatizing agent under conditions appropriate to produce derivatized polysaccharide particles, and (c) washing the crosslinked and derivatized particles.

In another aspect, the present invention is a method for making crosslinked derivatized polysaccharides, comprising: (a) contacting particles of a polysaccharide with a titanium compound in an aqueous medium under conditions appropriate to intra-particulately crosslink the particles; (b) reacting, prior to or subsequent to the step of contacting the particles of polysaccharide with the titanium compound, the particles of polysaccharide with a derivatizing agent under conditions appropriate to produce derivatized polysaccharide particles; (c) depolymerizing the particles of polysaccharide either (i) prior to or subsequent to the step of contacting the particles with the titanium compound or (ii) prior to or subsequent to the step of reacting the particles with the derivatizing agent, and (d) washing the crosslinked and derivatized particles.

In a further aspect, the present invention is a method for making crosslinked derivatized polysaccharides, comprising: (a) reacting the particles of polysaccharide with a derivatizing agent under conditions appropriate to produce derivatized polysaccharide particles; (b) washing the derivatized particles; (c) contacting, concurrently with or after the step of washing the derivatized particles, the derivatized particles with a titanium compound under conditions appropriate to intra-particulately crosslink the particles.

In one embodiment, the crosslinking of the titanium crosslinked polysaccharide is reversible and the kinetics of de-crosslinking are pH sensitive. The rate at which de-crosslinking of the polysaccharide occurs typically increases with decreasing pH. Typically, an aqueous dispersion of the crosslinked polysaccharide is maintained at a pH of greater than or equal to about 8, more typically greater than or equal to about 10, more typically greater than or equal to about 12, to maintain the polysaccharide in the form of water insoluble crosslinked particles and thus maintain the fluidity of the aqueous dispersion of the crosslinked polysaccharide and the crosslinking can typically be rapidly reversed by adjusting the pH of the aqueous medium to a value of less than or equal to about 8, more typically less than or equal to about 7 to de-crosslink the polysaccharide and allow dissolution of the de-crosslinked polysaccharide in the aqueous medium, typically to form a viscous aqueous solution of the polysaccharide in the aqueous medium.

In another aspect, the present invention is directed to derivatized polysaccharide made by the above-described method.

In yet another aspect, the present invention is directed to an aqueous composition comprising derivatized polysaccharide made by any of the above-described methods. In one embodiment, the aqueous composition comprises, based on 100 parts by weight (pbw) of the composition, from about 1 to about 30 pbw of crosslinked derivatized polysaccharides made by any of the aforementioned methods, from about 65 to about 95 pbw of water, and from about 5 to about 20 pbw of an electrolyte.
In another embodiment, the aqueous composition comprises, based on 100 parts by weight (pbw) of the composition, from about 1 to about 15 pbw crosslinked derivatized polysaccharides made by any of the aforementioned methods and from about 85 to about 98 pbw of water.

### DETAILED DESCRIPTION OF INVENTION AND PREFERRED EMBODIMENTS

As used herein, the terminology “aqueous medium” generally means a liquid medium that contains water, typically greater than or equal to 10 wt % water, more typically greater than or equal to 25 wt % water, even more typically greater than or equal to 50 wt % water and less than 90 wt %, more typically less than 75 wt %, and even more typically less than 50 wt % of one or more water miscible organic liquids, such as for example, an alcohol, such as ethanol or iso-propanol, and may, optionally contain one or more solutes dissolved in the aqueous medium. In one embodiment, the liquid portion of an aqueous medium consists essentially of water. As used herein the terminology “aqueous solution” refers more specifically to an aqueous medium that further comprises one or more solutes dissolved in the aqueous medium.

As used herein, the term “intra-particulately” means within each discrete particle of the polysaccharide and intra-particulate crosslinking thus refers to crosslinking between polysaccharide molecules of a discrete polysaccharide particle, typically between hydroxyl groups of such polysaccharide molecules, with no significant crosslinking between particles.

Suitable polysaccharides contain polymeric chains of saccharide constitutive units, and includes, for example, starches, celluloses, xanthans, such as xanthan gum, polyfructoses such as levan, and galactomannans such as guar gum, locust bean gum, and tara gum. The polysaccharides are not completely soluble in the aqueous medium and thus typically remain as a discrete solid phase dispersed in the aqueous medium.

In one embodiment, the polysaccharide is a starch or a cellulose.

In one embodiment, the polysaccharide is a polyfructose such as an inulin or levan. In one embodiment the polysaccharide is a levan, which is a polyfructose comprising 5-membered rings linked through β-2,6 bonds, with branching through β-2,1 bonds. Levan exhibits a glass transition temperature of 138° C. and is available in particulate form. At a weight average molecular weight of 1-2 million, the diameter of the densely-packed spherulitic particles is about 85 nm.

In one embodiment, the polysaccharide is a xanthan. Xanthans include but are not limited to xanthan gum and xanthan gel. Xanthan gum is a polysaccharide gum produced by Xathomonas campestris and contains D-glucose, D-mannose, D-glucuronic acid as the main hexose units, also contains pyruvate acid, and is partially acetylated.

In one embodiment, the polysaccharide is a galactomannan. Galactomannans are polysaccharides consisting mainly of the monosaccharides mannose and galactose. The mannose-elements form a chain consisting of many hundreds of (1,4)-β-D-mannopyranosyl-residues, with 1,6 linked-D-galactopyranosyl-residues at varying distances, dependent on the plant of origin. Naturally occurring galactomannans are available from numerous sources, including guar gum, guar splits, locust bean gum and tara gum. Additionally, galactomannans may also be obtained by classical synthetic routes, may be obtained by chemical modification of naturally occurring galactomannans or other generally known routes.

In one embodiment, the polysaccharide is a locust bean gum. Locust bean gum or carob bean gum is the refined endosperm of the seed of the carob tree, Ceratonia siliqua. The ratio of galactose to mannose for this type of gum is about 1:4.

In one embodiment, the polysaccharide is a tara gum. Tara gum is derived from the refined seed gum of the tara tree. The ratio of galactose to mannose is about 1:3.

In one embodiment, the polysaccharide is a guar gum. Guar gum, often called “guar flour” after grinding, refers to the mucilage found in the seed of the leguminous plant Cyamopsis tetragonolobus. The water soluble fraction (85%) is called “guaran,” which consists of linear chains of (1,4)-.beta.-D mannopyranosyl units—with α D-galactopyranosyl units attached by (1,6) linkages. The ratio of D-galactose to D-mannose in guaran is about 1:2. Guar gum may take the form of a whitish powder which is dispersible in hot or cold water. Native guar gum typically has a weight average molecular weight of between about 2,000,000 and about 5,000,000 grams per mole.

Guar seeds are composed of a pair of tough, non-brittle endosperm sections, hereafter referred to as “guar splits,” between which is sandwiched the brittle embryo (germ). After dehulling, the seeds are split, the germ (43-47% of the seed) is removed by screening, and the splits are ground. The ground splits are reported to contain about 78-82% galactomannan polysaccharide and minor amounts of some proteinaceous material, inorganic salts, water-insoluble gum, and cell membranes, as well as some residual seedcoat and embryo.

In one embodiment, the polysaccharide particles are guar splits are in the form of particles having an average particles size, as measured using a scale and an optical microscope, of from about 2 to about 5 millimeters

Suitable titanium compounds are those titanium (II), Titanium (III), titanium (IV), and titanium (VI) compounds that are soluble in the aqueous medium.

In one embodiment, the titanium compound is a titanium (IV) compound, that is, a titanium compound in which the titanium atoms of the compound are in the +4 oxidation state.

In one embodiment, the titanium compound is a titanium salt, more typically a water soluble titanium salt, such as titanium tetrachloride, titanium tetrabromide, or tetra amino titanate.

In one embodiment, the titanium compound comprises one or more titanium chelates. Suitable titanium chelates are commercially available and include, for example, titanium acetylacetonates, triethanolamine titanates, and titanium lactates

In one embodiment, the titanium compound comprises one or more titanium esters. Suitable titanium esters are commercially available and include, for example, n-butyl polytitanates, titanium tetrapropanolate, octyleneglycol titanates, tetra-n-butyl titanates, tetra-n-butyl titanates, tetra-2-ethylhexyl titanates, tetra-isopropyl titanate, and tetra-isopropyl titanate.

In one embodiment, the titanium compound is selected from diisopropyl di-triethanolamino titanate, titanate (2-), dihydroxy bis[2-hydroypropanato (2-)-O1, O2], ammonium salt, titanium acetylacetonate, titanium ortho ester, titanium (IV) chloride, and mixtures thereof

In one embodiment, the polysaccharide particles are contacted with the titanium compound in the aqueous medium under conditions appropriate to at least partially crosslink the hydroxyl groups of the respective guar splits particles. The crosslinking typically takes place intra-particulately, that is, within each discrete particle of guar splits, between the hydroxyl groups of the particle, without any significant crosslinking between guar splits particles.

In one embodiment, the polysaccharide particles are contacted with a solution of the titanium compound in the aqueous medium.

In one embodiment, aqueous medium comprises, based on 100 parts by weight (“pbw”) of the medium, from about 0.1 to about 15 pbw, more typically from about 0.5 to about 10 pbw, and even more typically from about 1 to about 5 pbw, of the titanium compound.

In one embodiment, the aqueous medium has a pH of from about 6 to about 14, more typically from about 6 to about 8.

In one embodiment, the aqueous medium and guar splits in step (a) comprise, based on 100 pbw of the combined amount of aqueous medium and polysaccharide particles, from about 20 to about 90 pbw, more typically from about 30 to about 60 pbw aqueous medium, and from about 10 to about 80 pbw, more typically, from about 40 to about 70 pbw, polysaccharide particles.

In one embodiment, polysaccharide particles are contacted with titanium compound in the aqueous medium at a temperature of from about 10 to about 90° C., more typically from about 15 to about 35° C., and even more typically, from about 20 to about 30° C.

In one embodiment, the polysaccharide particles are contacted with titanium compound in the aqueous medium for a time period of from about 1 minute to about 2 hours, more typically from about 5 minutes to about 60 minutes, and even more typically from about 15 to about 35 minutes.

Processes for making derivatives of polysaccharides are generally known. Typically, the polysaccharide is reacted with one or more derivatizing agents under appropriate reaction conditions to produce a guar polysaccharide having the desired substituent groups. Suitable derivatizing reagents are commercially available and typically contain a reactive functional group, such as an epoxy group, a chlorohydrin group, or an ethylenically unsaturated group, and at least one other substituent group, such as a cationic, nonionic or anionic substituent group, or a precursor of such a substituent group per molecule, wherein substituent group may be linked to the reactive functional group of the derivatizing agent by bivalent linking group, such as an alkylene or oxyalkylene group. Suitable cationic substituent groups include primary, secondary, or tertiary amino groups or quaternary ammonium, sulfonium, or phosphinium groups. Suitable nonionic substituent groups include hydroxyalkyl groups, such as hydroxypropyl groups. Suitable anionic groups include carboxyalkyl groups, such as carboxymethyl groups. The cationic, nonionic and/or anionic substituent groups may be introduced to the guar polysaccharide chains via a series of reactions or by simultaneous reactions with the respective appropriate derivatizing agents.

In one embodiment, the polysaccharide is reacted with an alkylene oxide derivatizing agent, such as ethylene oxide, propylene oxide, or butylene oxide, under known alkoxylation conditions to add hydroxyalkyl and/or poly(alkyleneoxy) substituent groups to the guar polysaccharide chains.

In one embodiment, the polysaccharide is reacted with a carboxylic acid derivatizing agent, such as sodium monochloroacetate, under known esterification conditions to add carboxyalkyl groups to the guar polysaccharide chains.

In one embodiment, the derivatizing agent comprises a cationic substituent group that comprises a cationic nitrogen radical, more typically, a quaternary ammonium radical. Typical quaternary ammonium radicals are trialkylammonium radicals, such as trimethylammonium radicals, triethylammonium radicals, tributylammonium radicals, aryldialkylammonium radicals, such as benzyldimethylammonium radicals, radicals, and ammonium radicals in which the nitrogen atom is a member of a ring structure, such as pyridinium radicals and imidazoline radicals, each in combination with a counterion, typically a chloride, bromide, or iodide counterion. In one embodiment, the cationic substituent group is linked to the reactive functional group of the cationizing agent by an alkylene or oxyalkylene linking group.

Suitable cationizing reagents include, for example:

epoxy-functional cationic nitrogen compounds, such as, for example, 2,3-epoxypropyltrimethylammonium chloride

chlorohydrin-functional cationic nitrogen compounds, such as, for example, 3-chloro-2-hydroxypropyl trimethylammonium chloride, 3-chloro-2-hydroxypropyl-lauryldimethylammonium chloride, 3-chloro-2-hydroxypropyl-stearyldimethylammonium chloride, and

vinyl-, or (meth)acrylamide-functional nitrogen compounds, such as methacrylamidopropyl trimethylammonium chloride.

In one embodiment, the polysaccharide is reacted with a chlorohydrin-functional quaternary ammonium compound in the presence of base, in an aqueous medium under relatively mild conditions, such as heating to a temperature of 40° C. to 70° C., to produce cationic guar splits, that is, derivatized guar splits having cationic functional groups.

In one embodiment, the polysaccharide comprises polysaccharide molecules having one or more substituent groups per molecule, wherein at least a portion of the substituent groups have been added by reaction of the polysaccharide with one or more derivatizing agents in an aqueous medium under appropriate reaction conditions.

In one embodiment, the derivatized polysaccharide comprises polysaccharide molecules having one or more substituent groups per molecule, wherein all or substantially all of the substituent groups have been added by reaction of the polysaccharide with one or more derivatizing agents in an aqueous medium under appropriate reaction conditions in one or more derivatization reaction steps.

In one embodiment, the derivatized polysaccharide comprises polysaccharide molecules having one or more substituent groups per molecule, wherein a first portion of the substituent groups have been added by reaction of the polysaccharide with one or more first derivatizing agents under appropriate reaction conditions in a first liquid medium, and a second portion of the substituent groups have been added by reaction of the polysaccharide with one or more second derivatizing agents in a second liquid medium under appropriate reaction conditions, wherein at least one of the first liquid medium and the second liquid medium is an aqueous medium.

In one embodiment, the first and second liquid media are each aqueous media. In one embodiment, the first and second liquid media are each the same aqueous medium. In an embodiment wherein the first and second liquid media are the same aqueous medium, the derivatization reactions with the first and second derivatizing agents may be conducted concurrently or in series in the same aqueous medium.

In one embodiment, one of the first and second liquid media is an aqueous medium and the other of the first and second liquid media is a liquid medium other than an aqueous medium and the derivatization reaction in the first liquid medium is conducted prior to the derivatization reaction in the second liquid medium. In one embodiment, the first liquid medium is an aqueous medium and the second liquid medium is a liquid medium other than an aqueous medium, such as, for example, a polar organic solvent, more typically, a water miscible organic solvent. In one embodiment, the first liquid medium is a liquid medium other than an aqueous medium and the second liquid medium is an aqueous medium.

In one embodiment, the derivatized polysaccharide is produced by reaction of guar splits with a derivatizing agent in an aqueous medium are in the form of water-swollen gum comprising from about 30 to 60 pbw, more typically from 30 to 50 pbw, guar splits and 40 to 70 pbw, more typically 50 to 70 pbw, water per 100 pbw of water-swollen gum.

In one embodiment, the step of contacting the derivatized polysaccharide with the aqueous wash medium is conducted subsequent to the step of by reaction of the polysaccharide with a derivatizing agent in an aqueous reaction medium under appropriate reaction conditions. In one embodiment, a water-swollen gum produced by reaction of guar splits with a derivatizing agent in an aqueous reaction medium is contacted with the aqueous wash medium.

In one embodiment, the derivatized polysaccharide is allowed to cool, typically to a temperature of less than or equal to about 50° C. prior to washing the derivatized guar splits.

In one embodiment, the derivatized polysaccharide is washed with the aqueous medium by contacting the derivatized polysaccharide with the aqueous medium and then physically separating the aqueous wash medium, in the form of an aqueous rinse solution, from the derivatized polysaccharide, wherein the contacting and separating steps taken together constitute one “wash step”.

One or more wash steps are conducted in any suitable process vessel. Each wash step may be conducted as a batch process, such as for example, in a stirred mixing vessel, or as a continuous process, such as for example, in a column wherein a stream of the derivatized guar splits is contacted with a co-current or counter-current stream of aqueous wash medium.

In one embodiment, the aqueous wash medium consists essentially of water, even more typically, of deionized water.

In one embodiment, derivatized polysaccharide is contacted with from about 2 to about 30 kilograms (“kg”), more typically from about 5 to about 20 kg, even more typically from about 5 to about 15 kg, of aqueous wash medium per each kilogram of derivatized polysaccharide solids per wash step.

In one embodiment, each wash step comprises contacting the derivatized polysaccharide with an aqueous wash medium for a contact time of up to about 30 minutes, more typically from about 30 seconds to about 15 minutes, even more typically from about 1 minute to about 8 minutes, per wash step.

The washed derivatized polysaccharide is separated from the aqueous wash medium by any suitable dewatering means such as for example, filtration and/or centrifugation. In one embodiment, the washed derivatized polysaccharide is separated from the wash liquid by centrifugation.

In one embodiment, dewatered titanium crosslinked derivatized guar splits have a water content of less than or equal to about 80 percent by weight (“wt %”), more typically less than or equal to about 70 wt %.

The dewatered derivatized polysaccharide particles are dried and ground to produce derivatized guar particles.

In one embodiment, the derivatized polysaccharide is dried by any suitable drying means, such as, for example, air drying, fluid bed drying, flash grinding, freeze drying, to a moisture content of less than or equal to about 20 wt %, more typically less than or equal to about 15 wt %.

In one embodiment, dried titanium crosslinked derivatized guar splits are ground by any suitable particle size reduction means, such as, for example, a grinding mill. In one embodiment the guar splits are simultaneously dried and ground in a “flash milling” procedure, wherein a stream of guar splits and a stream of heated air are simultaneously introduced into a grinding mill.

In one embodiment, the titanium crosslinked derivatized polysaccharide according to the present invention comprises a galactomannan polysaccharide that is substituted at one or more sites of the polysaccharide with a substituent group that is independently selected for each site from the group consisting of cationic substituent groups, nonionic substituent groups, and anionic substituent groups.

In one embodiment, the titanium crosslinked derivatized polysaccharide according to the present invention is selected from hydroxypropyl trimethylammonium guar, hydroxypropyl lauryldimethylammonium guar, hydroxypropyl stearyldimethylammonium guar, hydroxypropyl guar, carboxymethyl guar, guar with hydroxypropyl groups and hydroxypropyl trimethylammonium groups, and mixtures thereof.

In one embodiment, titanium crosslinked derivatized guar gum according to the present invention exhibits a total degree of substitution (“DS_{T}”) of from about 0.001 to about 3.0, wherein:

DS_{T} is the sum of the DS for cationic substituent groups (“DS_{cationic}”), the DS for nonionic substituent groups (“DS_{nonionic}”) and the DS for anionic substituent groups (“DS_{anionic}”),

DS_{cationic} is from 0 to about 3, more typically from about 0.001 to about 2.0, and even more typically from about 0.001 to about 1.0,

DS_{nonionic} is from 0 to 3.0, more typically from about 0.001 to about 2.5, and even more typically from about 0.001 to about 1.0, and

DS_{anionic} is from 0 to 3.0, more typically from about 0.001 to about 2.0.

In one embodiment, the molecular weight of the guar gum may be reduced by known means, for example, by treatment with a peroxide. The molecular weight reduction may be conducted prior to or subsequent to treatment with the titanium compound and prior to or subsequent to treatment with the derivatizing agent. In embodiment, the guar gum is treated to reduce its molecular subsequent to crosslinking the guar with the titanium compound and prior to treating the guar with a derivatizing agent.

In one embodiment, the boron content of the titanium crosslinked polysaccharide according to the present invention ranges from an undetectably low amount to less than about 50 ppm, more typically from an undetectably low amount to less than about 20 ppm, even more typically from an undetectably low amount to less than about 10 ppm, as measured by mass spectroscopy.

In one embodiment, particles of titanium crosslinked derivatized guar according to the present invention have an average mean particle size (“D₅₀”) of from about 10 to about 300 micrometers (“μm”), more typically from about 20 to about 200 μm, as measured by light scattering.

The crosslinks of the titanium crosslinked polysaccharide are reversible and the crosslinked polysaccharide tends to de-crosslink in the presence of water.

In one embodiment, the particles of titanium crosslinked polysaccharide are dispersed in water at a pH of greater than or equal to about 10, more typically greater than or equal to about 12, to form an aqueous dispersion of polysaccharide particles, typically comprising, based on 100 pbw of the dispersion, from about 2 to about 15 pbw, more typically 5 to 12 pbw, crosslinked polysaccharide particles and from about 85 to about 98 pbw, more typically from about 88 to about 95 pbw, water. At a pH of greater than or equal to about 10, more typically greater than or equal to about 12, the particles of crosslinked polysaccharide tend to de-crosslink slowly. The slow de-crosslinking allows the polysaccharide particles to remain at least partially crosslinked for some time, during which the aqueous dispersion tends to remain fluid and readily tractable and to not form a relatively intractable water swollen gel.

In one embodiment, a 10 wt % aqueous dispersion of particles of titanium crosslinked guar at a pH of greater than or equal to 10, more typically greater than or equal to about 12, remains fluid for a time period of greater than or equal to about 10 minutes, more typically, greater than or equal to about 15 minutes.

In one embodiment, the time period during which the aqueous dispersion of titanium crosslinked polysaccharide particles remains fluid is prolonged by reducing the concentration of crosslinked polysaccharide particles in the dispersion. In one embodiment, a 5 wt % dispersion of particles of titanium crosslinked guar at a pH of greater than or equal to 10, more typically greater than or equal to about 12, remains fluid for a time period of greater than or equal to about 15 minutes, more typically, greater than or equal to about 30 minutes.

In one embodiment, the time period during which a dispersion of titanium crosslinked polysaccharide particles in an aqueous medium remains fluid is prolonged by increasing the ionic strength of the aqueous medium. In one embodiment, an aqueous dispersion of particles of titanium crosslinked polysaccharide comprises, based on 100 pbw of the dispersion, from about 2 to about 15 pbw particles of titanium crosslinked polysaccharide and from about 85 to about 98 pbw water. In one embodiment, the aqueous dispersion of titanium crosslinked polysaccharide comprises, based on 100 pbw of the dispersion, from about 1 to about 30 pbw particles of titanium crosslinked polysaccharide, from about 65 to about 96 pbw water, and from about 5 to about 20 pbw of an electrolyte, typically an alkali metal or ammonium salt, more typically NaCl. In one embodiment, a 10 wt % dispersion of titanium crosslinked polysaccharide particles in an aqueous salt solution at a pH of greater than or equal to 10, more typically greater than or equal to about 12, remains fluid for a time period of greater than or equal to about 15 minutes, more typically greater than or equal to about 20 minutes, and even more typically for a time period of greater than 30 minutes.

At a pH above about 10, more typically greater than or equal to about 12, the crosslinking of the titanium crosslinked polysaccharide effectively provides a polymer network that is not readily swellable or soluble in water. The crosslinking is not permanent and can be rapidly reversed by adjusting the pH to provide non-crosslinked guar molecules. In the case of guar polysaccharide, the non-crosslinked guar molecules typically exhibit a weight average molecular weight of between about 500,000 and about 15,000,000 grams per mole, more typically a weight average molecular weight of from about 1,000,000 and about 10,000,000 grams per mole, most typically a weight average molecular weight of between about 2,000,000 and about 5,000,000 grams per mole, and are capable of being dissolved in water to produce a viscous aqueous solution. While not wishing to be bound by theory, it is believed that a portion of the polysaccharide remains in the form of water soluble complexes comprising two or more polysaccharide molecules linked by non-reversed titanium crosslinks and exhibiting a molecular weight that is higher, for example, by a factor of 2 to 5, than the molecular weight of the single non-crosslinked polysaccharide molecules.

A titanium crosslinked polysaccharide according to the present invention is capable of being de-crosslinked, that is, through release of the titanium crosslinks, and hydrated in an aqueous medium at a pH below about 10, more typically less than about 9, and even more typically less than or equal to about 7.

In one embodiment, titanium crosslinked guar particles are dispersed, typically in an amount of from about 0.1 to about 2 pbw guar particles per 100 pbw of the aqueous dispersion, prior to pH adjustment to reverse the crosslinking. In one embodiment, such a dispersion is formed by diluting a more concentrated aqueous dispersion of the titanium crosslinked derivatized guar particles.

In one embodiment, the pH of the aqueous medium is adjusted to a pH effective to allow rapid de-crosslinking and hydration of the derivatized guar by adding an effective amount of any suitable acid. In one embodiment, the acid is selected from citric acid, acetic acid, or hydrochloric acid. In one embodiment, the pH of the aqueous medium is adjusted by adding citric acid.

In one embodiment, the titanium crosslinked derivatized guar gum according to the present invention is capable of forming a substantially homogeneous solution at a pH of less than about 7 by stirring a mixture of water and 1 wt % of the guar gum for less than or equal to 8 hours, more typically, less than or equal to 4 hours, and even more typically, less than or equal to 2 hours. As used herein, the terminology “at least substantially homogeneous solution” means an aqueous mixture comprising the gum that appears, by visual examination, to be a single phase.

In one embodiment, a 1% solution of the de-crosslinked, hydrated derivatized guar gum according to the present invention in deionized water exhibits a viscosity of from about 50 to about 6000 centiPoise (“cP”), more typically from about 100 to about 5000 cP, as measured using a Brookfield RV viscometer (Brookfield Engineering Laboratories Inc. Middleboro, Mass.).

Polysaccharides that have been crosslinked according to the method of present invention are useful in personal care applications, such as, for example, shampoos, body washes, hand soaps, lotions, creams, conditioners, shaving products, facial washes, neutralizing shampoos, hair styling gels, personal wipes, and skin treatments.

In one embodiment, the personal care composition of the present invention comprises a polysaccharides that has been crosslinked according to the method of present invention and one or more “benefit agents” that is, materials known in the art that provide a personal care benefit, such as moisturizing or conditioning, to the user of the personal care composition, such as, for example, cleansing agents such as anionic surfactants, cationic surfactants, amphoteric surfactants, zwitterionic surfactants and non-ionic surfactants, as well as emollients, moisturizers, conditioners, polymers, vitamins, abrasives, UV absorbers, antimicrobial agents, anti-dandruff agents, fragrances, depigmentation agents, reflectants, thickening agents, detangling/wet combing agents, film forming polymers, humectants, amino acid agents, antimicrobial agents, allergy inhibitors, anti-acne agents, anti-aging agents, anti-wrinkling agents, antiseptics, analgesics, antitussives, antipruritics, local anesthetics, anti-hair loss agents, hair growth promoting agents, hair growth inhibitor agents, antihistamines, antiinfectives, inflammation inhibitors, anti-emetics, anticholinergics, vasoconstrictors, vasodilators, wound healing promoters, peptides, polypeptides and proteins, deodorants and anti-perspirants, medicament agents, hair softeners, tanning agents, skin lightening agents, depilating agents, shaving preparations, external analgesics, counterirritants, hemorrhoidals, insecticides, poison ivy products, poison oak products, burn products, anti-diaper rash agents, prickly heat agents, make-up preparations, amino acids and their derivatives, herbal extracts, retinoids, flavoids, sensates, anti-oxidants, hair lighteners, cell turnover enhancers, coloring agents, and mixtures thereof.

In one embodiment, the personal care composition of the present invention comprises an anti-dandruff active, such as, for example, pyridinethione salts, azoles, selenium sulfide, particulate sulfur, keratolytic agents, and mixtures thereof.

In one embodiment, the personal care composition according to the present invention is an aqueous composition that comprises, based on 100 pbw of the composition:
(a) greater than about 0.001 pbw, more typically from about 0.01 to about 0.8 pbw, and even more typically from about 0.1 to about 0.4 pbw, of a polysaccharide that has been crosslinked according to the method of present invention, and
(b) greater than about 1 pbw, typically from about 5 to about 20 pbw, and even more typically from about 10 to about 15 pbw, of a surfactant selected from cationic surfactants, anionic surfactants, amphoteric surfactants, zwitterionic surfactants, nonionic surfactants, and mixtures thereof.

In one embodiment, the polysaccharide component of the personal care composition has been at least partially de-crosslinked.

In one embodiment, the polysaccharide is a guar that has been crosslinked by the method of the present invention

In one embodiment, the polysaccharide is a derivatized guar that has been crosslinked by the method of the present invention.

In one embodiment, the personal care composition further includes a personal care benefit agent, more typically, a conditioning agent, an antidandruff agent, or a mixture thereof.

In one embodiment, the surfactant component (b) the personal care composition according to the present invention comprises a zwitterionic surfactant, more typically a zwitterionic surfactant selected from alkyl betaines and amidoalkylbetaines.

In one embodiment, the surfactant component (b) the personal care composition according to the present invention comprises a mixture of a zwitterionic surfactant, more typically a zwitterionic surfactant selected from alkyl betaines and amidoalkylbetaines, and an anionic surfactant, more typically selected from salts of alkyl sulfates and alkyl ether sulfates.

Cationic surfactants suitable for use in the personal care composition are well known in the art, and include, for example, quaternary ammonium surfactants and quaternary amine surfactants that are not only positively charged at the pH of the personal care composition, which generally is about pH 10 or lower, and soluble in the personal care composition. In one embodiment, the cationic surfactant comprises at least one n-acylamidopropyl dimethylamine oxide, such as cocamidopropylamine oxide.

Anionic surfactants suitable for use in the personal care composition are well known in the art, and include, for example, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and mixtures thereof.

Amphoteric surfactants suitable for use in the personal care composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. In one embodiment, the amphoteric surfactant comprises at least one compound selected from cocoamphoacetate, cocoamphodiacetate, lauroamphoacetate, and lauroamphodiacetate.

Zwitterionic surfactants suitable for use in the personal care composition are well known in the art, and include, for example, those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Specific examples of suitable Zwitterionic surfactants include alkyl betaines, such as cocodimethyl carboxymethyl betaine, lauryl dimethyl carboxymethyl betaine, lauryl dimethyl alpha-carboxy-ethyl betaine, cetyl dimethyl carboxymethyl betaine, lauryl bis-(2-hydroxy-ethyl)carboxy methyl betaine, stearyl bis-(2-hydroxy-propyl)carboxymethyl betaine, oleyl dimethyl gamma-carboxypropyl betaine, and lauryl bis-(2-hydroxypropyl)alpha-carboxyethyl betaine, amidopropyl betaines, and alkyl sultaines, such as cocodimethyl sulfopropyl betaine, stearyldimethyl sulfopropyl betaine, lauryl dimethyl sulfoethyl betaine, lauryl bis-(2-hydroxy-ethyl)sulfopropyl betaine, and alkylamidopropylhydroxy sultaines.

Nonionic surfactants suitable for use in the personal care composition are well known in the art, and include, for example, long chain alkyl glucosides having alkyl groups containing about 8 carbon atoms to about 22 carbon atoms, coconut fatty acid monoethanolamides such as cocamide MEA, coconut fatty acid diethanolamides, and mixtures thereof.

In one embodiment, the personal care composition further comprises a conditioning agent. Conditioning agents suitable for use in the personal care composition are well known in the art, and include any material which is used to give a particular conditioning benefit to hair and/or skin. In hair treatment compositions, suitable conditioning agents are those which deliver one or more benefits relating to shine, softness, antistatic properties, wet-handling, damage, manageability, and body. Conditioning agents useful in personal care compositions according to the present invention typically comprise a water insoluble, water dispersible, non-volatile, liquid that forms emulsified, liquid particles or are solubilized by the surfactant micelles, in an anionic surfactant component, as described above and include those conditioning agents characterized generally as silicones, such as silicone oils, cationic silicones, silicone gums, high refractive silicones, and silicone resins, and organic conditioning oils, such as hydrocarbon oils, polyolefins, and fatty esters.

Suitable silicone conditioning agents include silicone fluids, such as polyorganosiloxanes, for example, poly(alkylsiloxane)s, such as poly(dimethylsiloxane)s, cyclic poly(alkylsiloxane)s, such as mixtures of cyclomethicone tetramer, pentamer, and hexamer, and poly(alklarylsiloxane)s such as poly(methylphenylsiloxane)s.

Suitable organic conditioning oils for use as the conditioning agent in the personal care compositions include fatty esters, typically those having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.). Suitable fatty esters include, for example, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate. Other fatty esters suitable for use in the personal care compositions are those known as polyhydric alcohol esters. Such polyhydric alcohol esters include alkylene glycol esters. Still other fatty esters suitable for use in the personal care compositions are glycerides, including, but not limited to, mono-, di-, and tri-glycerides, preferably di- and tri-glycerides, more preferably triglycerides. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

In one embodiment, a derivatized guar gum according to the present invention provides improved delivery of a conditioning agent, more typically a silicone conditioning agent, onto and/or into the skin, hair, and/or nails.

The personal care composition according to the present invention may, optionally, further comprise other ingredients, in addition to benefit agents, such as, for example, preservatives such as benzyl alcohol, methyl paraben, propyl paraben, and imidazolidinyl urea, electrolytes, such as sodium chloride, sodium sulfate, and sodium citrate, thickeners, such as polyvinyl alcohol, pH adjusting agents such as citric acid and sodium hydroxide, pearlescent or opacifying agents, dyes, and sequestering agents, such as disodium ethylenediamine tetra-acetate.

### Example 1

Process water (about 26 pbw) and the titanium compound (about 0.4 pbw) were each charged to a reactor and the reactor contents were then mixed for 5 minutes. Guar splits (43 pbw) were then charged to the reactor and the reactor contents were then mixed for 30 minutes. A cationic derivatizing agent (Quat 188, about 17 pbw) was slowly added to the reactor and the reactor contents were then mixed for 30 minutes. NaOH (about 13 pbw) was then slowly added and the reactor contents were then mixed for 20 minutes. The reactor contents were then heated and maintained within a temperature range of 60-65° C. for 60 minutes. The reactor contents were then cooled to less than 40° C. and collected. The titanium crosslinked derivatized splits were washed two times for 2 minutes each with water at a ratio of 10 pbw water: 1 pbw splits, filtered, collected, and flash milled.

The viscosity of a 5% dispersion of the titanium crosslinked derivatized guar gum was measured at a pH of 12.95 grams DI water were adjusted to pH of 12 w/0.5 N NaOH, 5 grams of titanium crosslinked derivatized guar gum was added and stirred. The viscosity of the dispersion was measured at 25° C., using a Brookfield RV Viscometer equipped with a #3 spindle at 20 rpm. The viscosity results are set forth below in TABLE I in centiPoise (cP).

The pH of a 1% dispersion of the titanium crosslinked derivatized guar gum was adjusted to a value of 4 to reverse the crosslinking and the viscosity was then measured. 25 grams of a 5% dispersion of the titanium crosslinked derivatized guar gum were diluted to 125 g w/DI water, the pH of the resulting 1% dispersion was adjusted to 4 by adding 50% citric acid and the viscosity of the 1% dispersion was measured was measured at 25° C., using a Brookfield RV Viscometer, using a spindle and at a speed appropriate to the viscosity range, immediately after the pH adjustment and again after 2 hours and 24 hours of mixing. The viscosity results are set forth below in TABLE I in centiPoise (cP).

TABLE I

| | | 5% Dispersion* | | | 1% Dispersion** | | | Initial | 2 hrs |
|---|---|---|---|---|---|---|---|---|---|
| | Titanium | (cPs) | | | (cPs) | | | Visc | Visc |
| EX | Compound | 5 min | 15 min | 30 min | 5 min | 120 min | 24 hr | (%) | (%) |
| 1A | diisopropyl di- | 260 | 1050 | 2680 | 2440 | 3280 | 4140 | 59% | 79% |
| | triethanolamino | | | | | | | | |
| | titanate | | | | | | | | |
| | (TYZOR ® TE, | | | | | | | | |
| | DuPont) | | | | | | | | |
| 1B | titanate (2-), | 215 | — | 3700 | 2100 | 3150 | 3710 | 57% | 85% |
| | dihydroxy bis [2- | | | | | | | | |
| | hydroypropanato | | | | | | | | |
| | (2-)-O1, O2], | | | | | | | | |
| | ammonium salt | | | | | | | | |
| | (TYZOR LA, | | | | | | | | |
| | DuPont) | | | | | | | | |
| 1C | titanium | 150 | — | 2000 | 1505 | 2360 | 3000 | 50% | 79% |
| | acetylacetonate | | | | | | | | |
| | (TYZOR AA75, | | | | | | | | |
| | DuPont) | | | | | | | | |
| 1D | titanium (IV) | 500 | — | 1600 | 750 | 2260 | 3050 | 25% | 74% |
| | chloride | | | | | | | | |
| 1E | titanium ortho | 1550 | gel | — | 2660 | 3035 | 3610 | 74% | 84% |
| | ester | | | | | | | | |
| | (TYZOR 131, | | | | | | | | |
| | DuPont) | | | | | | | | |
| C1 | sodium tetra | <20 | <20 | <20 | — | 2970 | 3060 | — | 97% |
| | borate | | | | | | | | |

*95 grams DI water adjusted to pH = 12 w/ 0.5 N NaOH, add 5 grams product stir. Measure viscosity 25 C, RV#3, 20 rpm
**25 grams of 5% dispersion diluted to 125 g w/ DI water, lowered pH = 4 w/ 50% citric acid, measured viscosity 25 C, RV#3, 20 rpm

### Example 2

The titanium crosslinked guar of Example 2 was made in a manner analogous to that described above in regard to Example 1A and was de-crosslinked by diluting 25 grams of 5% guar dispersion to 125 g w/deionized water, adjusting the pH to the value indicated in TABLE II below with the acid indicated in TABLE II below and then measuring the viscosity resulting composition as described above in regard to Examples 1A-1E, that is, at 25° C. using a Brookfield RV viscometer, using a spindle and at a speed appropriate to the viscosity range, at the time intervals following pH adjustment indicated in TABLE II below.

TABLE II

| | | | | | | | | Viscosity (cP) | | |
|---|---|---|---|---|---|---|---|---|---|---|
| | | Viscosity (cPs) | | | Viscosity (cPs) | | | at pH = 12 | | |
| | | at pH = 5 | | | pH = 7 | | | (Control) | | |
| EX | Acid | 0 min | 2 h | 24 h | 0 min | 2 h | 24 h | 0 min | 2 h | 24 h |
| 2 | Hal | 90 | 1050 | 2100 | 50 | 400 | 760 | 5 | 10 | PS |
| | acetic | 350 | 1230 | 1950 | 50 | 570 | 1050 | | | |
| | citric | 1000 | 2450 | 2850 | 1000 | 2700 | 2900 | | | |

“PS” = phase separated

The results provided in Table II above indicate that adjusting the pH of titanium crosslinked guar dispersion with citric acid reversed the crosslink's of the titanium crosslinked guar more quickly and efficiently than acetic acid or Hal.

### Example 3

Titanium crosslinked guar made in a manner analogous to that described above in regard to Example 1A, except varying the amount of titanium compound used and the number of washes used, as noted in TABLE III below, was used as an ingredient in the conditioning shampoos of Examples 3A, 3B, 3C, and 3D. In each case a shampoo was prepared by combining the listed components in a mixing vessel and mixing.

| | pbw/100 pbw |
|---|---|
| Component | shampoo |
| Surfactant blend (34.6 wt % deionized water, 7.2 wt % | 90.9 |
| MiraTaine BETC30 (30.74 wt % active) cocamidopropyl | |
| betaine surfactant, 58.1 wt % Empicol ESB-3M (26.5% | |
| active) sodium lauryl ether sulfate surfactant, 0.055 wt % | |
| isothiazolinone biocide) | |
| silicone conditioning agent “dimethicone”, an aqueous | 1.5 |
| polydimethylsiloxane emulsion (65% active ingredient, | |
| droplet size of about 0.6 μm, Mirasil DM 500,000 | |
| emulsion, Rhodia) | |
| guar premix (5% guar in deionized water, pH adjusted to | 6 |
| 12 and mixed for 15 minutes) | |
| NaCl | 1.6 |

After addition of the listed ingredients, the pH was checked and adjusted to pH 6.0-6.5, if needed, using a citric acid or NaOH solution.

The shampoos of Comparative Examples C3A and C3B were made in a manner analogous to that used to make Examples 3A, 3B, 3C, and 3D, except that a boron crosslinked guar was substituted for the titanium crosslinked guar, analogous to that of Example C1 was used as the guar component.

The efficiency with which each of the shampoos was able to deposit the silicone conditioning agent on hair was evaluated. Silicone deposition efficiency of shampoos was measured on Virgin Medium Brown Caucasian Hair (hair tress weight: 4.5 grams, length below epoxy blue clip: 20 cm) supplied by IHIP (International Hair Importers & Products Inc.). Two measurements were done per shampoo to derive the mean value and standard deviation. The method includes the following 4 steps: (1) pre-treatment of the hair tresses with a 10% SLES (sodium lauryl ether sulfate) solution, (2) treatment of the hair tresses with the shampoo, (3) dimethicone extraction using tetrahydrofuran (“THF”), and (4) quantifying the extracted dimethicone by GPC, each described in greater detail below.
(1) Hair tresses were each pre-treated with a 10% sodium laureth sulfate (SLES) solution by: (a) wetting each of the hair tresses under the running water (water flow rate of 150 milliters/second, water temperature of 38° C.) for 1 minute, (b) applying 3 ml of a 10% SLES solution along each hair tress by hand, and then (c) rinsing the tress under the running water for 1 minute.
(2) The pre-treated tresses were then treated with one of the conditioning shampoos of the Examples as follows: (a) weighing out 450 mg quantity of shampoo, (b) rolling the hair tress around one of the experimenter's fingers and drawing the tress through the quantity of shampoo, (c) hand massaging the hair with the shampoo for 45 seconds, making sure that the shampoo was distributed evenly across the tress, and then (d) rinsing the tress under running water (water flow rate of 150 milliters/second, water temperature of 38° C.) for 30 seconds, (e) stripping off excess water from the tress by pulling the tress through the experimenter's middle finger and forefinger, and (f) leaving the tress to dry and equilibrate overnight in a controlled environment (21° C., 50% relative humidity).
(3) The silicone conditioning agent was extracted from each of the treated tresses with THF as follows: (a) Introducing the hair tress into a tared 250 ml polyethylene bottle while maintaining the mounting tab of the tress outside the bottle, (b) cutting the hair just below the mounting tab and recording the amount of hair introduced in the bottle, (c) introducing about 100 milliliters THF into the bottle, (d) capping the bottle, (e) agitating the bottle on an agitation table for 24 hours at 200 rpm, (f) transferring, under a hood, the THF extract solution from the bottle into a 150 milliliter evaporating dish, and (g) leaving the dish under the hood at maximum ventilation rate for 24 hours to evaporate the THF.
(4) The amount of extracted silicone conditioning agent was quantified by: (a) taring the evaporating dish capped with a watch glass, (b) introducing, under the hood, about 4 m milliliters of THF into the evaporating dish, (c) using a spatula, re-dissolving the dimethicone in the evaporating dish, (d) once the silicone was re-dissolved, weighing the evaporating dish capped with the watch glass and recording the amount of THF introduced, (e) transferring the dimethicone solution with a syringe into a 2 milliliter vial and capping the vial, and (f) determining the dimethicone concentration of the solution in the vial by GPC.

The amount of dimethicone deposited on hair, Q, expressed in parts per million (ppm, μg of dimethicone per g of hair) was then calculated as follows:

$$Q = \frac{{C}_{dimethicone} \times {m}_{THF}}{{m}_{hair}}$$

wherein:
C_{dimethicone} is the dimethicone concentration in the GPC vial expressed in ppm (μg dimethicone per gram of THF),
m_{THF} the amount of THF, expressed in grams, used to re-dissolve the dimethicone in the evaporating dish, and
mₕₐᵢᵣ, the amount of hair, expressed in grams, introduced in the polyethylene bottle.

The deposition efficiency was calculated as follows:

$$Y ( \% ) = \frac{Q \times {m}_{hair}}{10 \times φ \times {m}_{shampoo}}$$

wherein:

Q is the amount of silicone deposited onto hair (expressed in ppm),

mₕₐᵢᵣ is the amount of hair, expressed in grams, introduced in the polyethylene bottle,

φ is the weight fraction of dimethicone in active in the shampoo formulation (φ=0.01 for the examples considered here) and

mₛₕₐₘₚₒₒ is the amount of shampoo, expressed in milligrams, used to treat hair (here, mₛₕₐₘₚₒₒ˜450 mg).

Results are set forth below in TABLE III.

TABLE III

| | | | | amount | |
|---|---|---|---|---|---|
| | | | | deposited | |
| | | deposition | standard | on hair in | standard |
| EX | | yield | deviation | ppm | deviation |
| 3A | 1% diisopropyl di-triethanolamino titanate | 59% | 2% | 598 | 20 |
| | (TYZOR ® TE, DuPont) - 1 wash | | | | |
| 3B | 1% diisopropyl di-triethanolamino titanate | 63% | 1% | 632 | 14 |
| | (TYZOR ® TE, DuPont) - 2 washes | | | | |
| 3C | 2% diisopropyl di-triethanolamino titanate | 59% | 6% | 595 | 56 |
| | (TYZOR ® TE, DuPont) - 1 wash | | | | |
| 3D | 2% diisopropyl di-triethanolamino titanate | 65% | 0.1% | 655 | 9 |
| | (TYZOR ® TE, DuPont) - 2 washes | | | | |
| C3A | borated reference lot 1 | 52% | 3% | 524 | 17 |
| C3B | borated reference lot 2 | 52% | 2% | 530 | 12 |

The results in TABLE III indicate that the shampoos containing the titanium crosslinked guars exhibited significantly improved deposition of the conditioning agent onto the hair tresses compared to the analogous shampoos containing the boron crosslinked guars.

### Example 4

The titanium crosslinked guar of Example 4 was made in a manner analogous to that described above in regard to Example 1A, except varying the amount of titanium compound and the number of washes used, as noted in TABLE IV below, was used to prepare a 10% aqueous dispersion.

The titanium crosslinked guar of Example 4 was used to prepare two aqueous dispersions, one in deionized water and one in an aqueous 10% NaCl solution as follows: 108 grams of DI water or 108 grams of a 10% NaCl solution were adjusted to pH 12 with 20% NaOH. 12 grams of the titanium crosslinked guar of Example 4 was added and stirred. The viscosity of each of the dispersions was measured as a function of time at 25° C., using a Brookfield RV Viscometer at 20 rpm. Results are set forth below in TABLE IV as the time (in minutes) required to achieve a viscosity of 5000 centipoise.

TABLE IV

| | | Time in min to reach | |
|---|---|---|---|
| | | 5000 cP @ 20 rpm | |
| Ex | Description | Deionized Water | 10% NaCl |
| 4 | 1% diisopropyl di- | 13 | 30 |
| | triethanolamino | | |
| | titanate | | |
| | (TYZOR ® TE, DuPont), | | |
| | 1 wash | | |

The results in TABLE IV indicate that dispersing the titanium crosslinked guar in salted water helps in keeping the dispersion flowable for a longer time.

1. A method for making crosslinked derivatized polysaccharides, comprising:
(a) contacting particles of a polysaccharide with a titanium compound in an aqueous medium under conditions appropriate to intra-particulately crosslink the particles;
(b) reacting, prior to or subsequent to the step of contacting the particles of polysaccharide with the titanium compound, the particles of polysaccharide with a derivatizing agent under conditions appropriate to produce derivatized polysaccharide particles; and
(c) washing the crosslinked and derivatized particles.

2. The method of claim 1 wherein step (b) comprises reacting, after the step of contacting the particles of polysaccharide with the titanium compound, the particles of polysaccharide with a derivatizing agent under conditions appropriate to produce derivatized polysaccharide particles.

3. The method of claim 1 wherein step (b) comprises reacting, prior to the step of contacting the particles of polysaccharide with the titanium compound, the particles of polysaccharide with a derivatizing agent under conditions appropriate to produce derivatized polysaccharide particles.

4. The method of claim 1 wherein the titanium compound is selected from the group consisting of titanium (II), titanium (III), titanium (IV), titanium (VI) compounds and mixtures thereof.

5. The method of claim 1 wherein the titanium compound is selected from the group consisting of a titanium salt, a titanium chelate, a titanium ester and mixtures thereof.

6. The method of claim 1 wherein the titanium compound is selected from the group consisting of titanium tetrachloride, titanium tetrabromide, tetra amino titanate, titanium acetylacetonate, triethanolamine titanate, titanium lactate, n-butyl polytitanate, titanium tetrapropanolate, octyleneglycol titanate, tetra-n-butyl titanate, tetra-n-butyl titanate, tetra-2-ethylhexyl titanate, tetra-isopropyl titanate tetra-isopropyl titanate, diisopropyl di-triethanolamino titanate, titanium ortho ester, titanium (IV) chloride and mixtures thereof.

7. A crosslinked derivatized polysaccharide composition comprising, based on 100 parts by weight (pbw) of the composition, from about 1 to about 30 pbw of crosslinked derivatized polysaccharides made by the method of claim 1, from about 65 to about 95 pbw of water, and from about 5 to about 20 pbw of an electrolyte.

8. The polysaccharide composition of claim 7 wherein the electrolyte is selected from the group consisting of an alkali metal, ammonium salt, sodium chloride, sodium citrate, sodium sulfate and mixtures thereof.

9. A crosslinked derivatized polysaccharide composition comprising, based on 100 parts by weight (pbw) of the composition, from about 1 to about 15 pbw crosslinked derivatized polysaccharides made by the method of claim 1 and from about 85 to about 98 pbw of water.

10. A method for making crosslinked derivatized polysaccharides, comprising:
(a) contacting particles of a polysaccharide with a titanium compound in an aqueous medium under conditions appropriate to intra-particulately crosslink the particles;
(b) reacting, prior to or subsequent to the step of contacting the particles of polysaccharide with the titanium compound, the particles of polysaccharide with a derivatizing agent under conditions appropriate to produce derivatized polysaccharide particles;
(c) depolymerizing the particles of polysaccharide either (i) prior to or subsequent to the step of contacting the particles with the titanium compound or (ii) prior to or subsequent to the step of reacting the particles with the derivatizing agent; and
(d) washing the crosslinked and derivatized particles.

11. The method of claim 10 wherein step (b) comprises reacting, after the step of contacting the particles of polysaccharide with the titanium compound, the particles of polysaccharide with a derivatizing agent under conditions appropriate to produce derivatized polysaccharide particles.

12. The method of claim 10 wherein step (b) comprises reacting, prior to the step of contacting the particles of polysaccharide with the titanium compound, the particles of polysaccharide with a derivatizing agent under conditions appropriate to produce derivatized polysaccharide particles.

13. The method of claim 10 wherein the titanium compound is selected from the group consisting of titanium (II), titanium (III), titanium (IV), titanium (VI) compounds and mixtures thereof.

14. The method of claim 10 wherein the titanium compound is selected from the group consisting of a titanium salt, a titanium chelate, a titanium ester and mixtures thereof.

15. The method of claim 10 wherein the titanium compound is selected from the group consisting of titanium tetrachloride, titanium tetrabromide, tetra amino titanate, titanium acetylacetonate, triethanolamine titanate, titanium lactate, n-butyl polytitanate, titanium tetrapropanolate, octyleneglycol titanate, tetra-n-butyl titanate, tetra-n-butyl titanate, tetra-2-ethylhexyl titanate, tetra-isopropyl titanate tetra-isopropyl titanate, diisopropyl di-triethanolamino titanate, titanium ortho ester, titanium (IV) chloride and mixtures thereof.

16. A crosslinked derivatized polysaccharide composition comprising, based on 100 parts by weight (pbw) of the composition, from about 1 to about 30 pbw of crosslinked derivatized polysaccharides made by the method of claim 10, from about 65 to about 95 pbw of water, and from about 5 to about 20 pbw of an electrolyte.

17. The polysaccharide composition of claim 16 wherein the electrolyte is selected from the group consisting of an alkali metal, ammonium salt, sodium chloride, sodium citrate, sodium sulfate and mixtures thereof.

18. A method for making crosslinked derivatized polysaccharides, comprising:
(a) reacting the particles of polysaccharide with a derivatizing agent under conditions appropriate to produce derivatized polysaccharide particles;
(b) washing the derivatized particles;
(c) contacting, concurrently with or after the step of washing the derivatized particles, the derivatized particles with a titanium compound under conditions appropriate to intra-particulately crosslink the particles.

19. The method of claim 18 wherein the titanium compound is selected from the group consisting of titanium (II), titanium (III), titanium (IV), titanium (VI) compounds and mixtures thereof.

20. The method of claim 18 wherein the titanium compound is selected from the group consisting of a titanium salt, a titanium chelate, a titanium ester and mixtures thereof.

21. The method of claim 18 wherein the titanium compound is selected from the group consisting of titanium tetrachloride, titanium tetrabromide, tetra amino titanate, titanium acetylacetonate, triethanolamine titanate, titanium lactate, n-butyl polytitanate, titanium tetrapropanolate, octyleneglycol titanate, tetra-n-butyl titanate, tetra-n-butyl titanate, tetra-2-ethylhexyl titanate, tetra-isopropyl titanate tetra-isopropyl titanate, diisopropyl di-triethanolamino titanate, titanium ortho ester, titanium (IV) chloride and mixtures thereof.
